(19) United States
(12) Patent Application Publication
(10) Pub. No.: US 2017/0050045 A1
(43) Pub. Date: Feb. 23, 2017

(54) PHOTODYNAMIC THERAPY USING IN SITU NONLINEAR PHOTON UPCONVERSION OF NIR LIGHT BY BIOLOGICAL MEDIUM

(71) Applicant: The Research Foundation for The State University Of New York, Amherst, NY (US)

(72) Inventors: Paras N. PRASAD, Williamsville, NY (US); Artem PLISS, Amherst, NY (US); Aliaksandr KACHYNSKI, Tonawanda, NY (US); Tymish OHULCHANSKYY, Amherst, NY (US); Andrey N. KUZMIN, E. Amherst, NY (US)

(21) Appl. No.: 15/307,548

(22) PCT Filed: Apr. 28, 2015
(86) PCT No.: PCT/US2015/027928

§ 371 Date: Oct. 28, 2016

Related U.S. Application Data
(60) Provisional application No. 61/985,259, filed on Apr. 28, 2014.

(51) Int. Cl. A61N 5/06; A61K 31/409

(52) U.S. Cl. CPC A61N 5/062; A61K 31/409; A61N 2005/0659; A61N 2005/0662; A61N 2005/067

(57) ABSTRACT

Photodynamic therapy methods using near infrared light and visible-light-absorbing photosensitizers and methods of generating visible light in an individual. The methods use upconverted incident near infrared light, for example, to excite the photosensitizer or facilitate drug delivery. The methods can be carried out on humans and non-human animals.

### CROSS-REFERENCE TO RELATED APPLICATIONS

This application claims priority to U.S. provisional patent application no. 61/985,259, filed Apr. 28, 2014, the disclosure of which is incorporated herein by reference.

### STATEMENT REGARDING FEDERALLY SPONSORED RESEARCH

This invention was made with government support under contract no. FA9550-11-C-1012 awarded by the Air Force Office of Scientific Research. The government has certain rights in the invention.

### FIELD OF THE DISCLOSURE

The present disclosure generally relates to photodynamic therapy methods using near infrared (NIR) electromagnetic radiation. More particularly, the present disclosure relates to photodynamic therapy methods using upconverted NIR electromagnetic radiation and photosensitizers excited by visible light.

### BACKGROUND OF THE DISCLOSURE

Photodynamic therapy (PDT) has been employed to fight cancerous tumors and other diseases for almost three decades. The predominant pathway for this phototherapy involves light induced generation of highly cytotoxic singlet oxygen (¹O₂) by energy transfer from a photoexcited sensitizer molecule, often called PDT drug or PDT agent, for destruction of diseased tissue in human body. The PDT agents are accumulated in the tumors or other diseased sites. In the absence of light, the “dark toxicity” of the photosensitizer usually remains very low which prevents tissue damage to unexposed and unintended sites. PDT potentials are very broad, covering from mouth, throat, lung, intestinal and gallbladder cancers, to eye, skin and connective tissue diseases. A major limitation of PDT is insufficient propagation of light through the tissue, which hinders the treatment of remote tissues.

The scope of PDT applications can be significantly improved if new near IR (NIR) absorbing photosensitizers with optical and tumor-localizing properties superior to the so-called first generation photosensitizer, Photofrin, can be synthesized. Since NIR lies within the biological window of maximum optical transparency, the use of NIR light for phototherapy allows deep tissue penetration, thus enabling the treatment of remote or thick tumor.

The utilization of photosensitizers with multi-photon, and in particular, two-photon absorption in the NIR spectral range with subsequent energy transfer to the singlet oxygen for deep targeting and high resolution PDT treatment demonstrates one such advanced technology. This concept of two-photon photodynamic therapy led to an effort for development of photosensitizers that can efficiently be excited by strong two-photon absorption. A major limitation of two-photon PDT is that it is a resonant nonlinear process, thus usable wavelengths being limited to the region of two-photon absorption of a PDT agent. Since photosensitizers, capable of producing singlet oxygen, typically have low two-photon cross-sections, modification of the photosensitizer structure to obtain higher two-photon absorption cross-section is necessary for two-photon PDT to be successful. This may affect efficiency of singlet oxygen generation and/or pharmacokinetics of the PDT drug. Alternatively, conjugation (or intraparticle co-localization) of the two-photon absorbing dye with photosensitizer is requisite for excitation of the PDT agent through the energy transfer from two-photon absorbing moieties.

Another approach uses up-converting agents (inorganic nanocrystals) containing rare-earth-ions with multiple f-to-f transitions to convert deeply penetrating NIR light by sequential multiphoton absorption to visible emission, which then excites the PDT agent. A main issue with this approach is the narrow and weak NIR absorption of the rare-earth ions, thus producing low up-conversion efficiency. Besides, co-localized delivery of nanoparticles and PDT agent should be provided. The pharmacokinetics of the photosensitizing drug is affected by their combination with upconverters.

### BRIEF SUMMARY OF THE DISCLOSURE

The present disclosure provides PDT methods using NIR incident light and visible-light-excited photosensitizers. In the methods, singlet oxygen and/or reactive oxygen species are generated in a localized volume of an individual. In an embodiment, a method of generating singlet oxygen and/or reactive oxygen species in a localized volume of an individual comprises: a) exposing the individual to incident coherent pulsed electromagnetic energy having a wavelength between 700 nm and 1.4 microns; and b) exposing the individual to incident coherent pulsed electromagnetic energy having a wavelength between 700 nm and 1.4 microns and a repetition rate of from 100 MHz to 1 Hz, where a secondary electromagnetic energy having a wavelength of 350 to 700 nm is produced in the localized volume of the individual and the photosensitizing agent is excited by the secondary electromagnetic energy resulting in generation of singlet oxygen and/or reactive oxygen species in at least a portion of the localized volume of the individual. For example, the incident coherent light has an average power density of from 1×10⁶ W/cm² to 5×10⁷ W/cm². For example, the localized volume of the individual at least 50 microns below a surface of the individual exposed to the ambient atmposhere.

The methods can be carried out on a variety of individuals. The methods are suitable for human and non-human animals.

The photosensitizing agent can be administered to the individual by methods known in the art. For example, the photosensitizing agent is administered systemically (e.g., orally or by intravenous delivery) or locally to a desired area of an individual.

The photosensitizing agent has an absorption band maximum having a wavelength of from 350 nm to 700 nm, including all integer wavelength values and ranges therebetween. The photosensitizing compound produces singlet oxygen and/or reactive oxygen species when excited by visible light.

The incident light (i.e., electromagnetic radiation) is coherent, pulsed electromagnetic radiation. The incident light has a wavelength of from 700 nm to 1.4 microns, including all nm values and ranges therebetween. In various examples, the incident coherent electromagnetic energy is provided by a single laser or a first laser providing a first incident coherent electromagnetic energy and a second laser providing a second incident coherent electromagnetic energy and the first coherent electromagnetic energy and the second coherent electromagnetic energy are synchronized in time and overlapped in space.

The incident light is exposed to the individual through a surface of the individual that is exposed to the ambient atmosphere, a mucosal surface of the individual, a surface of the respiratory tract of the individual, a surface of the gastrointestinal tract of the individual, or a surface of the reproductive tract of the individual. The incident light is pulsed. The incident light is provided to a localized volume of the individual. The localized volume of the individual comprises one or more tissue components. Examples of suitable tissue components include collagen, lipids, proteins, RNA, DNA, and combinations thereof.

The incident light has a power density at least sufficient to produce upconverted light having an intensity sufficient to excite at least a portion of the photosensitizer present in the localized volume so that singlet oxygen and/or reactive oxygen species are produced in at least a portion of the localized volume or nearby (e.g., adjacent) tissue(s).

### BRIEF DESCRIPTION OF THE FIGURES

FIGS. 1a-1c. Representative Jablonski diagrams for nonlinear optical interactions utilized for optical signal generation for subsequent PDT excitation.

FIG. 2. Representative example of experimental nonlinear optical set up.

FIGS. 3a-3b. FIG. 3a is representative nonlinear excitation lay-out diagram. FIG. 3b shows PDT procedure diagram.

FIGS. 4a-4b. FIG. 4a is a fluorescence spectra of an example of a photosensitizer showing that the CARS/FWM excite the photosensitizer at 665 nm, increasing the photosensitizer fluorescence intensity. FIG. 4b is a fluorescence spectra of an example of a photosensitizer showing that the SHG from polymerized collagen gels excites the photosensitizer at 400 nm, increasing the photosensitizer fluorescence intensity.

FIG. 5. Control phototoxicity study data in absence of photosensitizer.

FIGS. 6a-6b. FIGS. 6a and 6b. show examples of in situ PDT treatment data using light up-converted from NIR to visible by FWM/CARS for example of photosensitizer.

FIGS. 7a-7b. FIGS. 7a and 7b show examples of in situ PDT treatment data using light up-converted from NIR to visible by SHG for example of photosensitizer.

### DETAILED DESCRIPTION OF THE DISCLOSURE

An object of the present disclosure is to provide photodynamic therapy (PDT) methods based on the in situ generation of visible electromagnetic radiation (also referred to herein at visible light). The PDT methods use near infrared (NIR) incident electromagnetic raditation (also referred to herein as NIR incident light) and visible-light-excited photosensitizers.

PDT is a medical treatment of cancer and other diseases initiated when light absorbed by a therapy agent (photosensitizer), generates reactive oxygen and/or reactive oxygen species to affect the diseased tissues. Provided herein are PDT methods, which by means of nonlinear optical interactions of incident intense near infrared (NIR) light (e.g., laser radiation) with natural biomedium (e.g., mammalian biomedium), that in situ produce light at the wavelength falling within the intense one-photon absorption band of the photosensitizer to effect PDT. Application of NIR radiation, followed by in situ up-conversion to visible light, provides deep tissue penetration for PDT, thus addressing a major hurdle in the current treatment of remote and thick tissues. The methods were demonstrated using a commercial photosensitizer, Chlorin e6. For example, efficient PDT drug activation with NIR laser radiation is accomplished by in situ nonlinear-optical up-conversion using: (i) Second Harmonic Generation in collagen and/or (ii) Four-Wave Mixing, including Coherent anti-Stokes Raman Scattering (CARS), a third-order nonlinear optical process produced by a natural biological constituents, e.g., intracellular biomolecules such as intracellular proteins, lipids, nucleic acids, and aquatic biological environment (e.g., water).

Elevated content of lipids is a common feature for a broad group of solid tumors including adrenal, mammary, brain, and others. The following are examples of solid tumors: 1) adrenal tumors, also known as adrenal masses; 2) pancreatic neuroendocrine tumors; 3) lipid-rich carcinoma (a rare breast cancer with an aggressive clinical course and poor prognosis); 4) brain metastases originated from broad types of tumors including lung, melanoma and mammary carcinoma; 5) colon adenocarcinoma; and 6) adipose tumors. It is worth noting that content of lipids may vary from one patient to another. For many types of tumors high content of lipids correlates with a poor clinical outcome.

A specific type of protein (collagen) accumulates in many tumors. Examples include mammary neoplasia, rectal cancer and bone tumors. Collagen can generate signal for excitation of photosensitizer both in CARS (proteins) and SHG modality. Also, nucleic acids, such as RNA, are usually elevated in tumors.

The use of NIR radiation for light energy transport through a biomedium and generation of the photoexcitation light in situ within the malignancy, allows minimization of losses of excitation light (due to scattering and absorption) on the way to reaching the photosensitizer, permitting deeper treatment as compared to conventional PDT as well as two-photon PDT. Furthermore, in situ light conversion of incident NIR radiation to the excitation light occurs in a localized area (could be smaller than the diffraction limit because of the nonlinear conversion nature), essentially increasing spatial resolution and thus specificity of PDT treatment which could be very valuable for ophthalmologic and neurological applications.

In an aspect, the present disclosure provides PDT methods. The methods use near infrared incident light and visible-light-excited photosensitizers. In the methods, singlet oxygen and/or reactive oxygen species are generated in a localized volume of an individual. In an embodiment, only visible-light-excited photosensitizers are used in the method.

In an embodiment, a method of photodynamic therapy comprises: a. administering a photosensitizer (e.g., a PDT agent) to a patient afflicted with a tumor; b. applying to the tumor a first laser wave (SHG mode). In a further embodiment, the method may further comprise: c. applying to the tumor a second laser wave; and d. applying to the tumor a third laser wave, synchronized in time and superposed in space with second wave (CARS mode).

In an embodiment, a method of photodynamic therapy comprises: a. administering a PDT agent to a patient afflicted with a tumor; b. applying to the tumor a first laser wave; and c. applying to the tumor a second laser wave, synchronized in time and superposed in space with first wave (CARS mode).

In an embodiment, the method of photodynamic therapy comprises: a. administering a PDT agent to a patient afflicted with a tumor; b. applying to the tumor a first laser wave of about 780-820 nm, having a pulse width in the range between 5 ps and 20 ps and a corresponding repetition rate about 50-100 MHz (SHG mode). In a further embodiment, the method may further comprise: c. applying to the tumor a second laser wave of about 780-820 nm, having a pulse width in the range between 5 ps and 20 ps with a corresponding repetition rate about 50-100 MHz; and d. applying to the tumor a third laser wave of 1064 nm, synchronized in time and superposed in space with second wave (CARS mode).

In an embodiment, the method of photodynamic therapy comprises: a. administering a PDT agent to a patient afflicted with a tumor; b. applying to the tumor a first laser wave of about 780-820 nm, having a pulse width in the range between 5 ps and 20 ps with a corresponding repetition rate about 50-100 MHz; and c. applying to the tumor a second laser wave of 1064 nm, synchronized in time and superposed in space with first wave (CARS mode).

Throughout this application, “tumor” is used to refer to any malignancy. The use of the singular encompasses the plural throughout this application.

In an embodiment, a method of generating singlet oxygen and/or reactive oxygen species in a localized volume of an individual comprises: a) administering to the individual a photosensitizing agent having an absorption band maximum in the wavelength range of from 350 to 700 nm; and b) exposing the individual to incident coherent pulsed electromagnetic energy having a wavelength between 700 nm and 1.4 microns, where a secondary electromagnetic energy having a wavelength of 350 to 700 nm is produced in the localized volume of the individual and the photosensitizing agent is excited by the secondary electromagnetic energy resulting in generation of singlet oxygen and/or reactive oxygen species in at least a portion of the localized volume of the individual. For example, the pulse duration is from 1 femtosecond to 100 nanoseconds, including all values and ranges therebetween, and/or the repetition rate is from 100 MHz to 1 Hz, including all values and ranges therebetween.

The methods can be carried out on a variety of individuals (also referred to herein a patient). The methods are suitable for human and non-human animals. Accordingly, the methods can be used for human and veterinary purposes. For example, the individual is a human or a non-human animal. Examples of non-human animals include non-human mammals.

The photosensitizing agent can be administered to the individual by methods known in the art. For example, the photosensitizing agent is administered systemically (e.g., orally or by intravenous delivery) or locally to a desired area of an individual. The photosensitizing agent is administered concomitantly with or prior to exposing the individual to the incident light. The photosensitizing agent may be absorbed and/or accumulate in a specific area (e.g., a specific tissue) of the individual.

Photosensitizers are administered in “effective amounts,” i.e., at a dosage that facilitates the desired biological effects (e.g., absorption and/or accumulation of the photosensitizer in the target, such as a specific tissue or portion thereof, and/or blood vessel and/or tissue destruction). A useful dosage of a photosensitizer in the methods depends, for example, on a variety of properties of the activating light (e.g., wavelength, energy density, intensity), the optical properties of the target tissue, and properties of the photosensitizer. The upper and lower dosage limits depend on the type of photosensitizer used, and these limits are generally known for a variety of photosensitizers. In addition, the photosensitizer dosimetry can be determined empirically by those skilled in the art. A factor in determining the dosage per administration is the number of administrations to be given prior to light treatment. Thus, in the methods, the dosage can be lower than typically used with a given photosensitizer so that the total of all fractionated doses can be the same or lower than the standard dose for a given photosensitizer.

The photosensitizing agent has an absorption band maximum having a wavelength of from 350 nm to 700 nm, including all integer wavelength values and ranges therebetween. The photosensitizing compound produces singlet oxygen and/or reactive oxygen species when excited by visible light. Without intending to be bound by any particular theory, it is considered that singlet oxygen and/or reactive oxygen species are formed by energy transfer from the first excited singlet state of the photosensitizer. The photosensitizing agent can be a photodynamic therapy agent or drug. Combinations of two or more photosensitizing agents can be used. Suitable photosensitizing agents are known in the art. Suitable photosensitizing agents and drugs are commercially available and can be made using methods known in the art. For example, the photosensitizer is a Type 1 or Type 2 PDT drug. Examples of suitable photosensitizing agents include porphyrins, bacterioporphyrins, corrins, chlorins, bacteriochlorines, bacteriochlorophylls, corphins, phtalocyanins, azadipyrromethenes, and metal complexes thereof. In an embodiment, the photosensitizer is Chlorin E6 (aspartyl chlorin (excitation wavelengths centered at ˜400 nm and ˜667 nm)), Photochlor® (HPPH (excitation wavelengths centered at ˜400 nm and ˜665 nm)), Photofrin® (porfimer sodium (excitation wavelengths centered at ˜400 nm and ˜630 nm)), Visudyne® (verteporfin), Levulan® (δ-aminolevulinic acid), Foscan® (temoporfin), Metvix®/Visonac® (methyl aminolevulinate), Hexvix®/Cysview®/Lumacan® (hexaminolevulinate), Laserphyrin® (mono-L-aspartyl chlorin e6, Antrin (motexafin lutetium), Photosens, Photrex® (rostaporfin), Cevira®, BF-200 ALA, Amphinex® (tetraphenyl chlorin disulfonate), an azadipyrromethene, or a combination thereof. In an embodiment, the cross-section of the photosensitizing agent is insufficient for two-photon absorption.

The incident light (i.e., electromagnetic radiation) is coherent, pulsed electromagnetic radiation. The incident light is also referred to herein as a laser wave. The incident light has a wavelength of from 700 nm to 1.4 microns, including all nm values and ranges therebetween. In an embodiment, the incident light has a wavelength of 700 nm to 1 micron and/or 1.1 microns to 1.4 microns. Without intending to be bound by any particular theory, it is considered that interaction (e.g., by scattering mechanisms) of the incident electromagnetic radiation with a biological medium the individual (e.g., a tissue and/or one or more tissue components) is a nonlinear, non-resonant process that provides upconverted secondary electromagnetic radiation (i.e., light) having visible wavelengths in at least a portion of the localized volume of the individual. The upconverted light is produced by mechanisms such as, for example, Second Harmonic Generation (SHG) and/or Four-Wave Mixing, which includes Coherent anti-Stokes Raman Scattering (CARS) single harmonic generation.

The incident light is exposed to the individual through a surface of the individual that is exposed to the ambient atmosphere, a mucosal surface of the individual, a surface of the respiratory tract of the individual, a surface of the gastrointestinal tract of the individual, or a surface of the reproductive tract of the individual. For example, the incident light is exposed to a localized area of the individual through a surface of the skin of the individual, a surface of a lung, nasal cavity, throat, or windpipe, a surface of the esophagus, stomach, intestine, or rectum, or a surface of the uterus of the individual. In an embodiment, the individual is exposed to the incident NIR electromagnetic radiation without exposing the localized volume of the individual to the ambient atmosphere (e.g., without surgically exposing the localized volume of the individual).

The incident light is pulsed. For example, the incident light is pulsed laser light. In various embodiments, the pulse duration is from 1 femtosecond to 100 nanoseconds, including all integer values and ranges therebetween, and/or the repetition rate of from 100 MHz to 1 Hz, including all integer values and ranges therebetween. In various embodiments, the repetition rate is 100 kHz or less, 500 kHz or less, or 1 MHz or less.

The incident light may be focused. For example, the incident light is focused laser light. It is considered that focusing the incident light increases the efficiency of non-linear conversion. The incident light may be pulsed and focused. For example, the incident light is focused and pulsed laser light.

The incident light can be provided for exposure to the localized volume of the individual by one source or multiple sources (e.g., two or three sources). For example, the incident light is delivered a single laser, two lasers, or three lasers. The lasers can be fixed wavelength lasers or tunable lasers. Examples of suitable lasers include Ti-sapphire lasers, Optical Parametric Oscillators, Dye lasers, Lasers based on Rare-Earth ions, such as fiber lasers. The incident light can be delivered using a fiber (e.g., a delivery fiber). Suitable fibers are known in the art.

In an embodiment, the incident light is delivered from two sources. One or both of the light sources (e.g., lasers) can be tunable. In an embodiment, one laser is tunable and a second laser has a fixed wavelength. Without intending to be bound by any particular theory, secondary electromagnetic radiation having a desired wavelength or wavelengths can be generated. For example, by selecting one or more incident wavelengths of incident light (e.g., tuning one or more of the lasers) secondary electromagnetic radiation having a desired wavelength or wavelengths can be generated for a given localized volume of the individual.

Tuning of CARS to match specific resonance frequencies of proteins, lipids or nucleic acids, provides a mechanism for selective excitation of a photosensitizer associated with different structural elements of the treated tissue. In particular, using lipid CARS resonance can be very useful for PDT of many types of tumorigenic lesions known to accumulate lipids. For example, tuning the optical parametric oscillator (OPO) frequency to 812.6 or 818.0 nm aligns the resonance vibration frequencies to vibration bands of proteins (2930 cm⁻¹) or lipids (2840 cm⁻¹). Resonance (CARS) emission from RNA/DNA in the particular case of Stokes wave will be close to max of absorption band of Chlorin e6 in red range and corresponds to pump wave of 809 nm.

In the case where the incident light is delivered by two sources (e.g., two lasers), the light from the individual sources in location of the upconversion are synchronized in time and overlapped in space. For example, the incident light is provided by two lasers synchronized in time and space and the upconverted light is produced by Four-Wave Mixing and/or Coherent anti-Stokes Raman Scattering (CARS).

The incident light is provided to a localized volume of the individual. The localized volume of the individual comprises one or more tissue components. Examples of suitable tissue components include collagen, lipids, proteins, RNA, DNA, and combinations thereof. In an embodiment, the localized volume of the individual is not exposed the ambient atmosphere. In an embodiment, the localized volume does not comprise an exogenous tissue component.

The localized volume of the individual can include the skin of the individual or be beneath the skin of the individual (e.g., not include the skin of the individual) or beneath a mucosal surface of the individual. In an embodiment, the localized volume of the individual is a volume of the individual in which the photosensitizing agent is absorbed and/or accumulated (e.g., a specific tissue or portion thereof in which the photosensitizing agent is absorbed and/or accumulated). The localized volume can be below a surface of the individual that is exposed to the ambient atmosphere, a mucosal surface of the individual, a surface of the respiratory tract of the individual, a surface of the gastrointestinal tract of the individual, or a surface of the reproductive tract of the individual. In various examples, the localized volume is 2 cm or less, 4 cm or less, or 6 cm or less from a surface of the individual. In various embodiments, the localized volume is at least 50 microns, 100 microns, 200 microns, 500 microns, 1 mm, 5 mm, 10 mm, or 500 mm from a surface of the individual exposed to the ambient atmosphere, a mucosal surface of the individual, a surface of the respiratory tract of the individual, a surface of the gastrointestinal tract of the individual, or a surface of the reproductive tract of the individual.

The localized volume of the individual is an axially and laterally resolved spatial domain of the individual. The incident light may be focused over a range of focal lengths so the focal plane of the incident light is at a position within the localized volume of the individual. The focal length of the light may be varied and/or scanned over different parts of the individual so that the incident light is provided to more than one part of the of the localized volume of the individual or to multiple localized volumes of the individual.

Accordingly, the localized volume of the individual may include a structure that is a target for photodynamic therapy. The target can be biological complex and/or cellular or tissue structure. For example, the target is a cancerous tissue (e.g., a tumor), a tissue in blood vessels that occur in disorders characterized by hypervascularization or proliferation of neovascular networks, abnormal cells (or a tissue with abnormal cells), undesirable avascular tissue (e.g., hair follicles), tissue afflicted with a dermatological disease (e.g., psoriasis, actinic keratosis, haemangioma, and acne), or a wound. For example, at least a portion or all of the localized volume of the individual includes a tumor and/or other cancerous tissue structure. In another example, at least a portion or all of the localized volume includes tissue in blood vessels associated with hypervascularization or proliferation of neovascular networks, abnormal cells such as cancerous cells (or a tissue with abnormal cells), undesirable avascular tissue (e.g., hair follicles), tissue afflicted with a dermatological disease (e.g., psoriasis, actinic keratosis, haemangioma, and acne), and/or a wound. The localized volume may include normal cells (e.g., a tissue with normal cells) in addition to the aforementioned targets for photodynamic therapy.

In an embodiment, the localized volume of the individual comprises collagen and the upconverted light is produced by SHG. In an embodiment, the localized volume of the individual comprises collagen and at least one other tissue component. In an embodiment, the localized volume of the individual comprises collagen, lipids, proteins, RNA, DNA or a combination thereof and the upconverted light is produced by Four-Wave Mixing and/or, Coherent anti-Stokes Raman Scattering (CARS).

The incident light has a power density at least sufficient to produce upconverted light having an intensity sufficient to excite at least a portion of the photosensitizer present in the localized volume so that singlet oxygen and/or reactive oxygen species are produced in at least a portion of the localized volume or nearby (e.g., adjacent) tissue(s). For example, the power density of the incident light is up to 10⁹ W/cm². For example, the average power density of the incident light is from 10⁶ W/cm² to 5×10⁷ W/cm², including all integer W/cm² values and ranges therebetween. In an example, the average power density is at least 5×10⁵ W/cm². Without intending to be bound by any particular theory it is considered that the singlet oxygen and/or reactive oxygen species lead to localized cessation of cell proliferation, cell necrosis, and/or destruction of either or both the cells and surrounding vasculature in a target tissue in the localized volume or nearby (e.g., adjacent) tissue(s) (e.g., a tumor or portion thereof). In an embodiment, the method inhibits the growth of cells (e.g., abnormal cells such as cancerous cells) in the localized volume of the individual. In an embodiment, the method inhibits the growth of cells (e.g., abnormal cells such as cancerous cells) in the localized volume of the individual and in at least a portion of the area of the individual adjacent to the localized volume.

In another embodiment, the incident light is delivered from two light sources. For example, the incident light is delivered by/from two lasers, the pulse duration is from 1 picosecond to 100 nanoseconds, and the localized volume comprises lipids, proteins, RNA, DNA, and combinations thereof.

The methods may be carried out concomitantly with conventional two-photon photodynamic therapy. In an embodiment, the method further comprises administration of a two-photon photosensitizer. Suitable two-photon photosensitizers are known in the art.

The methods of the present disclosure can be carried out without the presence of special agents (which do not include photosensitizers as described herein) that upconvert the incident NIR light, such as, for example, inorganic nanoparticle upconverting agents (e.g., noble metal and metal oxide (e.g., ZnO) nanoparticles) and noble metals or metal oxides) or upconversion phosphors. In an embodiment, there are no detectible special agents in the localized volume and/or nearby (e.g., adjacent) tissue. In an embodiment, there are no exogenous special agents in the localized volume of the individual.

The steps of the methods described herein (e.g., in the various embodiments and examples) are sufficient to carry out the PDT methods and/or methods of generating visible light in a localized volume of an individual of the of the present disclosure. Thus, in an embodiment, a particular method consists essentially of a combination of the steps of a method disclosed herein. In another embodiment, a particular method consists of such steps.

The following examples are presented to illustrate the present disclosure. They are not intended to limiting in any manner.

### Example 1

This example describes an example of a method of the present disclosure.

Experimental Details. We selected a commercially available photosensitizer, Chlorin-e6, for demonstration of these new approaches. Three different types of nonlinear optical up-conversion mechanisms of PDT initiation were comparatively studied: 1) direct two-photon absorption (TPA) in the PDT agent, 2) second harmonic or sum frequency generation—SHG/SFG in collagens, with subsequent one-photon excitation of the PDT agent, and 3) Coherent anti-Stokes Raman Scattering (CARS) and associated four-wave mixing (FWM) produced by the natural intracellular macromolecules (proteins and lipids), with subsequent one-photon excitation of the PDT agent and energy transfer for single oxygen generation.

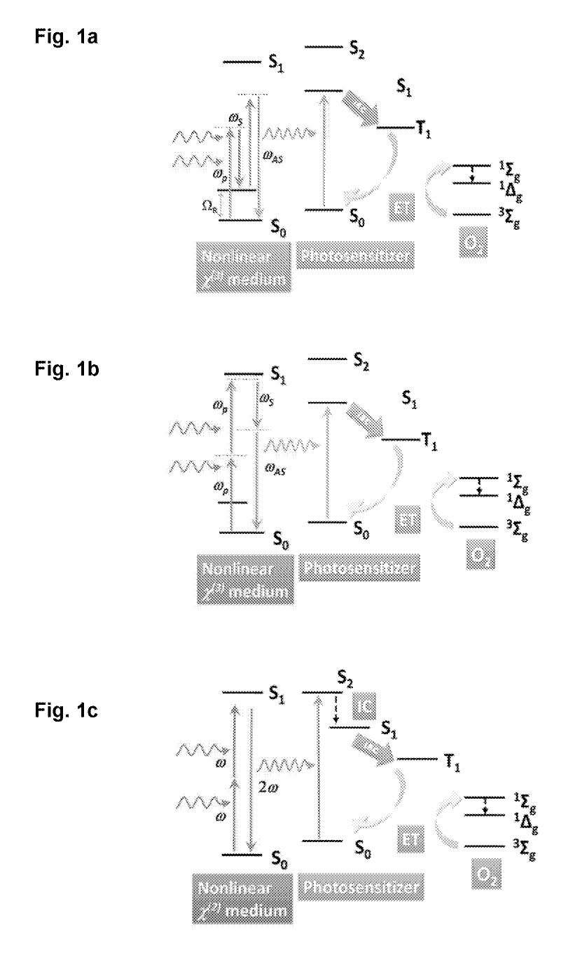

The diagrams for the nonlinear optical interactions utilized for optical signal generation for subsequent PDT excitation are presented in FIGS. 1a-1c. Chlorin-e6 is suited for all three nonlinear processes, it has two intense absorption bands peaked at ˜400 nm and ˜667 nm. Since singlet oxygen activation occurs through energy transfer from the first excited singlet state of the photosensitizer, the choice of excitation wavelength is not of particular importance as long as it fits into the absorption band. We perform a proof of concept of the technology, and discuss optimal optical experimental configurations and the incident light beams parameters for respective nonlinear optical processes for enhancement of the efficiency of PDT treatment.

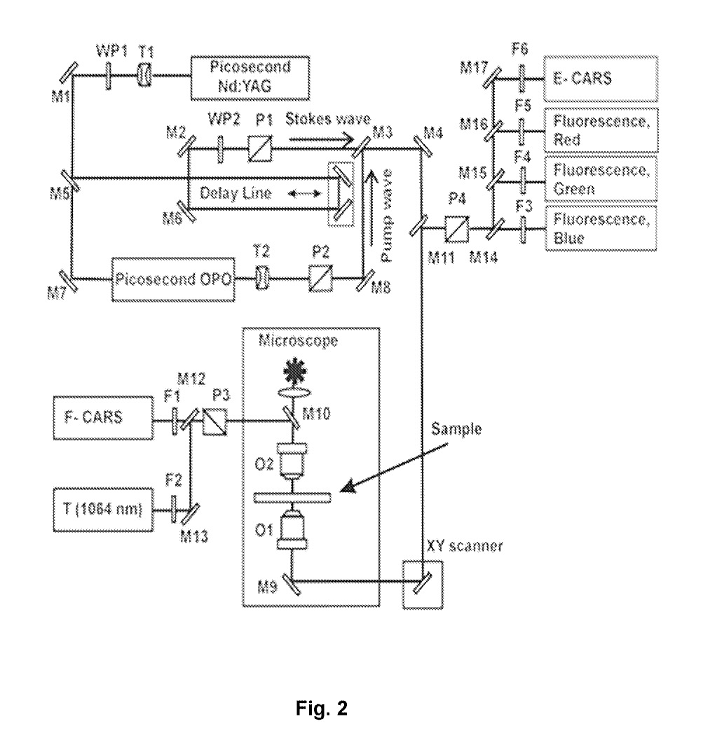

Materials and Methods. Optical setup. The experimental setup used for the present nonlinear optical excitation of PDT utilized a custom made laser scanning microscopy system with two excitation NIR laser sources. A simplified optical scheme is shown in FIG. 2. A picosecond Nd:YVO4 laser (picoTRAIN IC-10000/532-4000, HighQ Laser) with pulse width ˜5 ps and a repetition rate of 76 MHz was used as the source for the Stokes wave ωₛ at the fundamental output of 1064 nm as well as for synchronous pumping of a tunable optical parametric oscillator (OPO, Levante Emerald, APE) by using its 532 second harmonic output with a pulse width of ˜4 ps. The synchronously pumped OPO produces the pump/probe wave at ωₚ=ωₚᵤₘₚ=ω_{probe} in the tunable 670-990 nm range, for degenerate CARS/FWM interactions. The two picosecond laser waves were made coincident in time and space using a series of dichroic mirrors and delay line, and focused into the sample using Plan Apo VC 60× WI, 1.2 NA or Plan Fluor 20×0.50 NA Nikon objective lenses. Vibrationally resonant CARS and its satellite non-resonance FWM processes involve the pump/probe wave and the Stokes wave at frequencies ωₚ and ωₛ, respectively (see FIGS. 1a-1c). When the beating frequency ωₚ-ωₛ is tuned to be resonant with a vibrational mode of a selected molecular bond, the CARS signal together with its nonresonant electronic FWM background signal is detected at the anti-Stokes frequency of ωₐₛ=2ωₚ−ωₛ. Using our instrument setup, a sample can be excited and the imaged in the CARS/FWM mode in the vibrational frequency range of 900-3300 cm⁻¹ and simultaneously at 670-990 nm in the TPEF (two-photon excited fluorescence) or/and in the SHG mode. To separate CARS/FWM from the TPEF mode an adjustable time delay between the Stokes (Neodymium laser) and the pump (OPO) pulses was detuned to 5 ps by a computer controlled optical delay line. A XY galvano scanner (VM1000XY, GSI Lumonics) scanned the sample in the lateral focal plane with a resolution of 500×500 pixels at a rate of ˜1 frame/sec. Four photomultiplier tubes (PMT) provided (R928, R5108, Hamamtsu Photonics) detection of various signals. A computer control of interchangeable interference filters and 16 bits acquisition system allowed for multi-channel, wavelength selective, signals detection and processing. A custom-made software ensured locked in operation of the XY galvano scanner and the signal detection/processing electronics, allowing for simultaneous acquisition of four multi-modal digital images. Thus, this experimental setup enables concurrent operation of the system in the PDT excitation mode as well as in the imaging mode (CARS/FWM/fluorescence) which is highly suitable for PDT process monitoring. A Fiber coupled spectrometer, SpectraPro 2500 (Acton Research), was used for spectral monitoring. The final results of PDT interaction with the cell culture were monitored in a Leica SP2 confocal fluorescent microscopy setup using appropriate sample staining, with Calcein AM and Propidium Iodide fluorophores.

Tuning the OPO frequency to 812.6 or 818.0 nm was performed to align the resonance vibration frequencies to vibration bands of proteins (2930 cm⁻¹) or lipids (2840 cm⁻¹) and the CARS anti-Stokes emission frequency ωₐₛ=2ωₚ−ωₛ correspondingly to the wavelength of 657 nm or 665.4 nm, which overlap with the absorption band of photosensitizer Chlorin e6 in the visible range of the spectrum (670 nm maximum). In addition, the non-resonant electronic FWM signal at the same ωₐₛ frequencies as shown in the diagram in FIG. 1a,b will also contribute. Moreover, the incident excitation radiation ωₚ at 812.6 nm and 818.0 nm are closely matched with the wavelength of direct Two-Photon absorption band of photosensitizer Chlorin-e6. Thus the resulting PDT enhancement will be derived from the combined actions of CARS, FWM and TPA. The Second Harmonic signal of the ωₚ wave lies in the short wavelength range of spectrum around 400 nm which coincides with the most intensive Soret absorption peak of Chlorin e6 and could be used for efficient excitation of PDT.

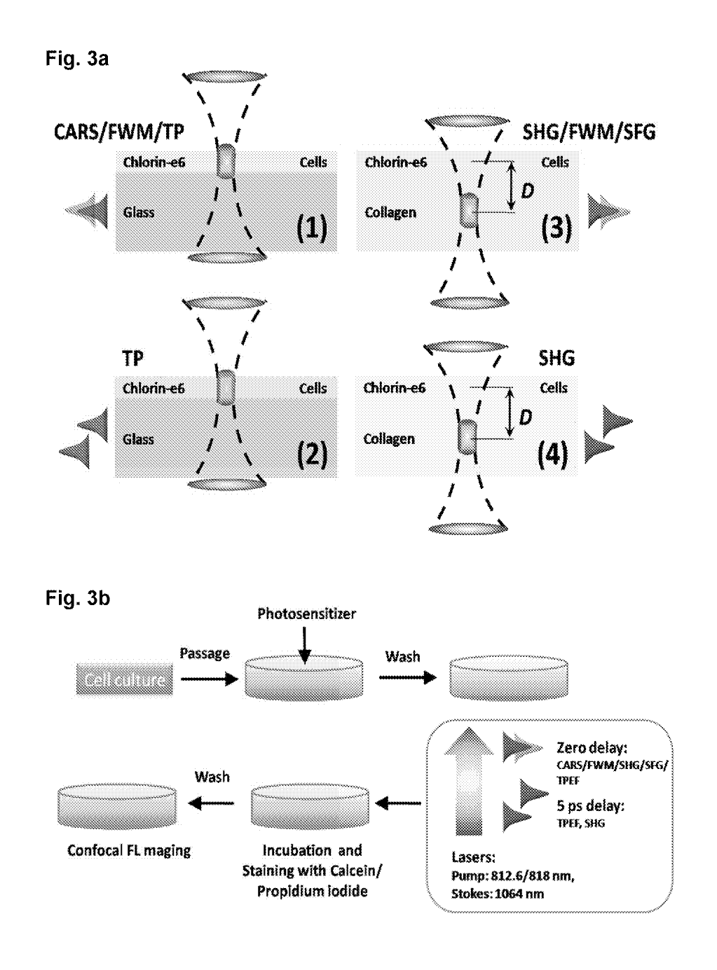

To verify the efficiency of the different nonlinear-optical conversion mechanisms for PDT excitation, cell cultured samples were used in different lay-outs permitting separation of the relative contribution of one specific nonlinear process. The nonlinear excitation lay-out diagram is shown in FIG. 3a. All experiments were made in a Petri dish through the bottom optical glass window in an inverted microscopy configuration. When cells were grown directly on the optical glass window, the pump and Stokes waves were synchronized with a zero delay and the beams were focused into the cell culture treated with the photosensitizer, CARS/FWM signals were generated by the natural intercellular biomolecules of proteins or lipids. In this case, the CARS/FWM conversion was also supplemented by the TPA process directly in the photosensitizer and a combination of CARS/FWM/TPA was obtained (see diagram 1 in FIG. 3a). To separate the TPA contribution, a pulse delay of ˜5 ps between the pump and the Stokes waves was introduced which disabled the CARS/FWM process. Therefore, with the pulse delay t, TPA of photosensitizer produced the dominant PDT effect (see diagram 2 in FIG. 3a). We used another lay-out in which cells were grown on a thin layer of polymerized collagen gel (ordered collagen structures). When the pump and Stokes laser beams were focused in collagen and had zero delay, SHG/FWM/SFG (sum frequency generation) nonlinear interactions occurred in the collagen layer (see diagram 3 in FIG. 3a). To selectively look at the SHG contribution, a pulse delay was introduced which disabled the FWM and SFG processes. The second harmonic signal generated by the collagen structure propagated to reach the cells treated with the photosensitizer, and efficiently absorbed by it to excite PDT (see diagram 4 in FIG. 3a). The dose of laser radiation delivered to the sample was estimated to be ˜60 J/cm² at 812.6 or 818.8 nm; ˜30 J/cm² at 1064 nm per scan, for CARS/FWM in proteins/lipids cellular environmental medium; and ˜60 J/cm² for SHG in polymerized collagen gels.

Cell culture, drug treatment and PDT design. The process of PDT was modeled on the cultured HeLa cells. FIG. 3b diagrammatically illustrates the PDT procedure. HeLa cells were grown in Advanced DMEM (Life Technologies), supplemented with 2.5% fetal calf serum (FBS) (Sigma, St. Louis, Mo.), 1% glutamax (Life Technologies), 1% Antibiotic Antimycotic Solution (Sigma) at 37° C. in a humidified atmosphere containing 5% CO₂. Prior to the incorporation of the photosensitizer and PDT experiments, cells were placed into glass-bottom dishes (MatTek, Ashland, Mass.). In the experiments involving SHG, the glass-bottom dishes were coated either with monomeric collagen (Sigma) or with collagen gels polymerized on the glass window. A stock solution of Chlorin-e6 was prepared in DMSO. The PDT process in the cultured cells was initiated by nonlinear optical excitation of the Chlorin-e6 photosensitizer, which was diluted to a final concentration of 90 μM in Advanced DMEM containing 15% FBS and 0.005% Tween 80. Cells were incubated with the drug for ninety minutes, thoroughly washed to remove unincorporated drug and subsequently used for optical exposure for PDT treatment.

Under these experimental conditions, the cells reproducibly exhibited similar fluorescence intensity levels of Chlorin-e6. During the laser irradiation, cells were maintained at 37° C. and 5% CO₂ in a Live-Cell incubator (Pathology Devices, Westminster, Md.) mounted on the microscope stage. Following the laser irradiation, the cells were incubated for 4 hours in a fresh regular medium at 37° C. At the next step of viability assessment, the cells were incubated for 1 hour in a serum-free MEM containing 1 μM of Calcein AM and 500 nM of propidium iodide (PI). Calcein AM is a cell permeable and non-fluorescent compound. Upon entering metabolically active cells, it is cleaved by intracellular esterases to yield a fluorescent dye (excitation/emission peaks are at 495/515 nm), after which it loses its ability to permeate the cell membrane, and is retained into the cell interior. Cells with low enzymatic activities or compromised integrity of membranes exhibit weak intensity or absence of the calcein signal. PI is a fluorescent nucleic acid stain (excitation/emission peaks are at 536/617 nm) that can permeate only through damaged membranes and is used as a selective marker of necrotic or late apoptotic cells. For detection of calcein and propidium iodide signals, cells were washed and then used for imaging with the Leica SP2 confocal microscope, equipped with an incubation chamber.

Results and Discussion. The light energy propagated through the medium is absorbed by Chlorin e6, either by two-photon or single-photon absorption mechanisms; the excess excitation energy is subsequently redistributed into a radiative (fluorescent) and a non-radiative triplet channel. The triplet excitation energy is then subsequently transferred to excite the triplet ground state of a nearby oxygen molecule to its singlet state. In our experiments, the fluorescence intensity of Chlorin e6 was chosen to serve as an indicator of the excitation efficiency of the photosynthesizer and, to the first approximation, of the efficiency of PDT.

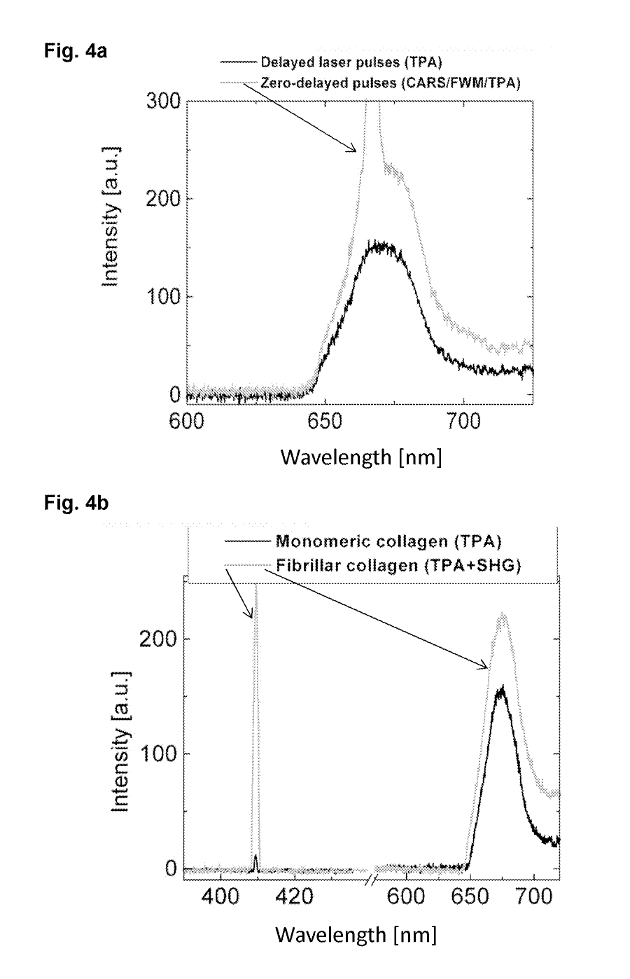

Chlorin e6 was diluted in the culture medium at a final concentration of 90 μM and its fluorescence intensity was measured in different experimental settings. In first series of experiments, Chlorin e6 was placed in the dish covered with a thin lipid layer, and was excited using dual laser coincided beams at 1064 nm (Stokes wave) and 818 nm (pump wave tuned to the vibrational resonance of the lipids) by focusing the beams on the lipid covered bottom of the dish. These laser pulses were either synchronized in time with zero delay for the CARS/FWM/TPA mode or applied with a 5 ps time delay, disabling CARS/FWM interactions in the lipid layer but retaining the TPA contribution. The emitted fluorescence intensity of Chlorin e6 was detected by the fiber coupled spectrometer. For the synchronized incident laser pulses with zero delay, the emitted fluorescence signal was higher as compared to the case of the laser pulses with a 5 ps delay for the same incident beam intensity (FIG. 4a). An enhancement of the Chlorin e6 fluorescence with zero delay laser pulses originated from additional single photon absorption of the nonlinear CARS/FWM signal produced by lipid layer at the anti-Stokes wavelength of 665 nm. This generated wavelength corresponds to the maximum of the Chlorin e6 absorption band (˜670 nm) and was efficiently absorbed by the photosensitizer, increasing the fluorescence emission. The CARS/FWM intensive peak at 665 nm overlaps with the fluorescence spectrum and is clearly seen in FIG. 4a. Also, it is worth noting that the CARS/FWM signal for lipids resonance in live cells is most probable to be reabsorbed by molecules of PDT drugs associated with membranes of cellular organelles (e.g. lysosomes, mitochondria), producing enhanced photodamage of these subcellular domains vital for cellular regulation. In a similar manner, protein vibration resonance with the CARS/FWM anti-Stokes wavelength at 657 nm can be applied to increase the Chlorin e6 fluorescence and enhance the PDT efficiency.

In the second series of experiments, we applied and studied SHG in collagen for the enhancement of Chlorin e6 excitation. In this experiment, the same excitation laser beam with 818 nm wavelength was focused in the sample. Chlorin e6 buffered solutions were placed on the top of either polymerized collagen gels known to produce a strong SHG on their quasi-ordered structure or on the top of monomeric collagen layers that represent relatively amorphous substance and produce only negligible SHG as shown in FIG. 4b. SHG wavelength of 409 nm corresponds well to the strongest Soret absorption band of Chlorin e6, and evidently contributes to excitation of the fluorophore (FIG. 4b). In the absence of any SHG, fluorescence offset in the FIG. 4b is produced by direct TPA in the buffered solution of the monomeric collagen.

Thus, our result confirms that in certain conditions, a series of nonlinear optical interactions (CARS/FWM and SHG) between the intense laser radiation and natural biological molecules and their structure can enable in situ enhancement of the photoexcitation of the PDT photosynthesizer.

The above concept was experimentally validated by modeling the PDT treatment in the live HeLa cells growing in ˜90% confluent monolayer cultures. The experimental nonlinear optical setup, shown in FIG. 2 was used for regulated irradiation of cell culture samples for excitation of PDT and for cell imaging.

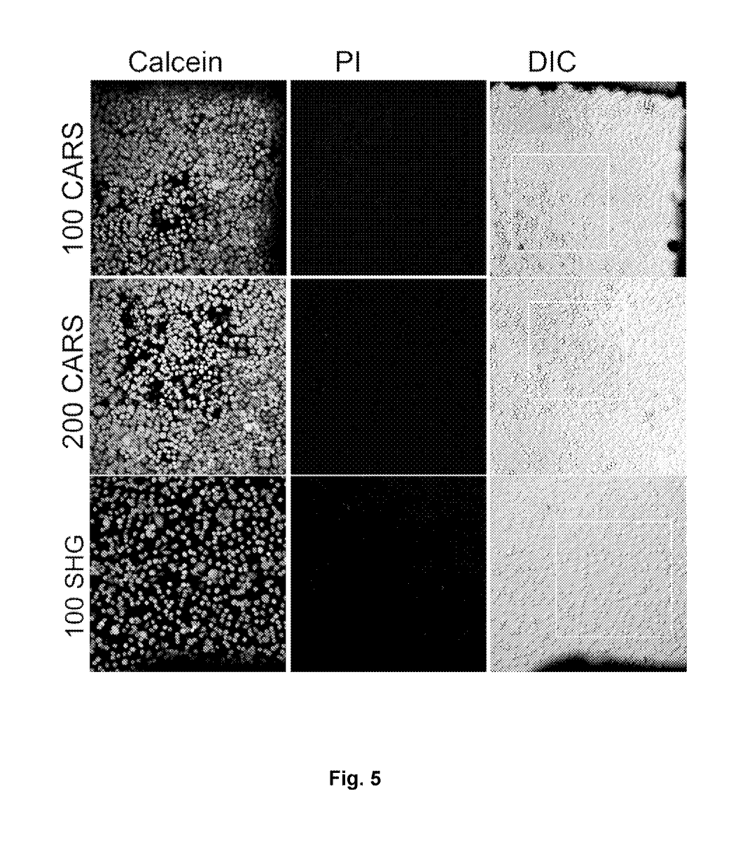

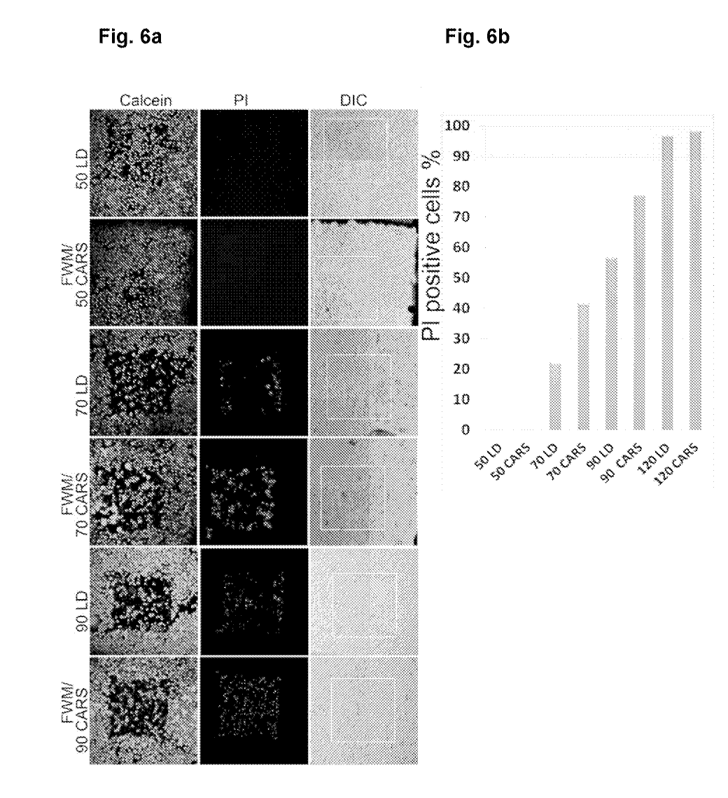

In order to characterize the cytotoxicity caused by the laser radiation itself, cells were incubated with calcein AM and Propidium Iodide (PI) as described in the materials and methods. The photosensitizer was not added to the sample processing in this experiment. The cells cultured in Petri dish were scanned with the focused dual laser beams at the pump and Stokes wavelengths with the synchronized zero pulse delay under operational power (see Materials and Methods) and incubated with the combination of these dyes. In the absence of the PDT drug, there were no signs of cytotoxicity due to interaction of the cultured cells with the picosecond pulses. The cells showed no visible change in the intensity of Calcein AM fluorescence and did not incorporate PI in response to the irradiation by up to 200 sequential laser scans (see confocal fluorescent images in FIG. 5). It is worth noting that a low cytotoxicity of nonlinear imaging is also consistent with our earlier reports. In the first series of experiments, we studied an in situ nonlinear-optical conversion of the incident laser radiation on the natural intracellular biomolecules of lipids by the resonance CARS/FWM/TPA mechanisms to initiate PDT. Cells grown on the glass window of the dish were treated with Chlorin e6 and irradiated by sequential series of scans of the dual beam laser pulses as described in Materials and Methods. The pump wave was adjusted to the wavelength of the vibration resonance of lipids (818 nm or 2840 cm⁻¹). Samples were scanned either in the conditions of time synchronized pump and Stokes laser pulses with zero delay to effect CARS/FWM/TPA or delayed pulses to trigger PDT by unaided TPA. FIGS. 6a-6b presents the PDT treatment data. Cellular fluorescence images of Calcein and PI staining, together with the DIC transmission light images for different scan numbers are shown. We observed that both nonlinear-optical conversion mechanisms induce PDT into the irradiated area. The cellular phototoxicity correlated with the irradiation dose. The increase of the laser scan number first diminished the fluorescence intensity of calcein signal pointing to decrease of cellular metabolic activity. Further increase of the irradiation dose led to rapid development of necrosis, as indicated by increasingly higher density of cells positively stained with PI (middle panel in the FIG. 6a).

In the PDT treatment, the cellular phototoxicity induced by in situ CARS/FWM/TPA nonlinear-optical mode was significantly higher than phototoxicity in the TPA mode for the same number of laser scans. FIG. 6b displays a diagram were the percentage of PI positive cells is plotted against the number of scans for the two studied nonlinear-optical modes averaged over four series of experiments. At the irradiation doze of ˜6300 J/cm² delivered to the cells by CARS/FWM/TPA nonlinear-optical excitation in 70 scans, we observed on the average 40% necrotic cells, as compared to 20% necrotic cells treated with the same number of scans in the TPA mode. Further increase of the scan numbers leads to saturation of damaged cells in both types of optical settings, although the higher efficiency of CARS/FWM/TPA is still notable with 90 scans (FIG. 6 a, b). We thus found a significantly higher efficiency and lower threshold of PDT treatment in the CARS/FWM/TPA mode as compared to the conventional TPA approach.

We also addressed the mechanism involving SHG conversion of the incident IR laser radiation for triggering of PDT. The practical value of this mechanism for single-photon excitation of the photosynthesizer is apparent from the fact that the stroma of a variety of solid tumors exhibits extensive deposits of collagen fibers (aggregated and not monomeric form). To study the PDT enhancement by SHG nonlinear-optical conversion in a collagen ordered structure, dishes were covered either with collagen fiber gels producing strong SHG signals (FIGS. 4a-b) or, in control, with monomeric (amorphous) collagen that generates negligible SHG. Cells were incubated on the top surface of both types of collagen substrates. For both settings, Chlorin e6 treated cells were irradiated with 818 nm laser focused in the collagen layer. As discussed above, the SHG signal at 409 nm falls into the intense Soret absorption band of Chlorin e6 and can trigger PDT.

In these experiments, PDT was triggered by first the generation of SHG in the collagen layer, subsequently propagated and absorbed by Chlorin e6 in the interiors of cultured cells growing on collagen gel surface. In addition, the photosynthesizer was concurrently excited by direct TPA due to the dimensions of the laser waist exceeding the thickness of collagen layer. Also, a contribution from weak collagen autofluorescence with a broad spectrum overlapping with Chlorin e6 absorption could play a certain role in PDT excitation.

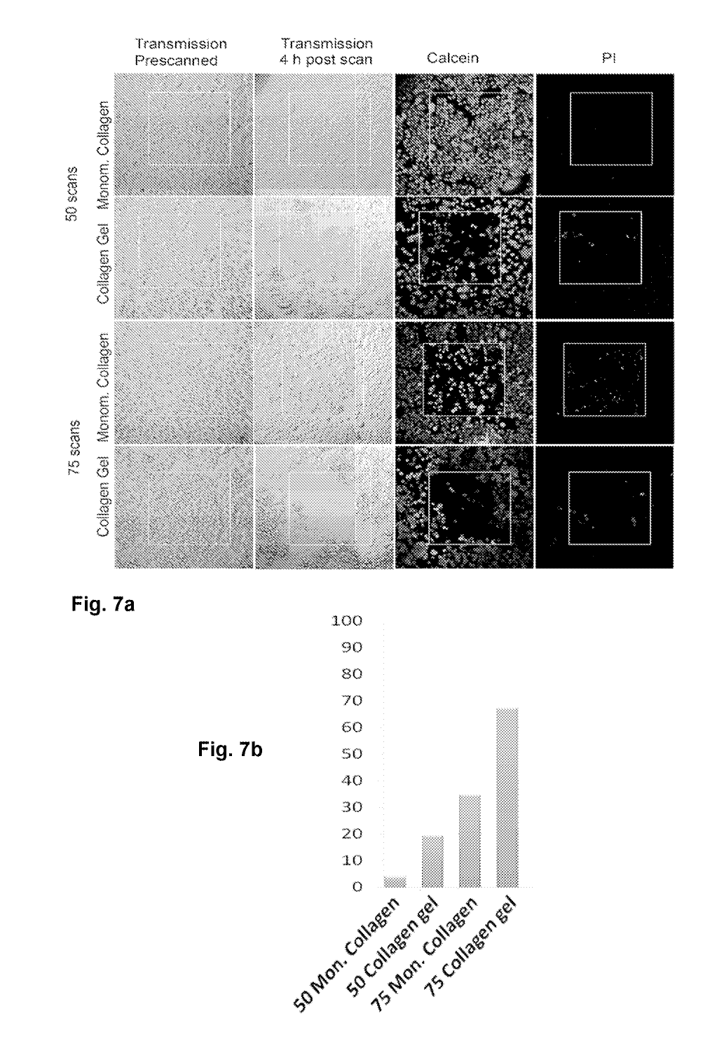

In the control experiments, by irradiating cells growing either on the monomeric or polymerized collagen substrates, we found no detectable cytotoxicity in the absence of PDT drugs (FIG. 5) irradiated by a single laser beam at 818 nm, even in hundred scans. The excitation with a single laser beam has been applied to this experiment to extract and identify the input of SHG nonlinear-optical conversion to PDT excitation, eliminating other nonlinear optical mechanisms such as resonance SFG, CARS, FWM and SHG from 1064 nm laser. The results of phototoxicity study are shown in FIGS. 7a-b. It is worth noting that the cells growing on the collagen substrates and treated with Chlorin e6 are more easily detached in response to laser irradiation, than treated cell growing directly on the uncoated glass surface. Therefore, for quantitative analysis of phototoxicity we counted both detached and PI positive cells as photodamaged.

Consistent with theoretical modeling, we found that the input of SHG, among other types of the nonlinear-optical conversion, appeared to be more significant for PDT excitation. In the sample with cells growing on the layers of fibrillar collagen, after 50 scans by 818 nm pulsed laser (corresponding radiation dose 3000 J/cm²), we observed a substantial diminishing of calcein signal intensity as well as detachment from the substrate, or positive PI staining of up to 50% of the cells. At the same irradiation dose, in contrast to the cells growing on the polymerized collagen, the cells on the monomeric collagen showed less than 5% cytotoxicity, as judged by counting of the detached and PI positive cells. Furthermore, we found little or no difference in the intensity of calcein staining at this irradiation dose in cells growing on monomeric collagen (FIG. 7a, b). The tenfold difference in phototoxicity between the two above experimental groups confirms the high efficiency of SHG nonlinear-optical conversion for Chlorin e6 PDT action. When the irradiation dose was increased to 75 scans corresponding to ˜4500/cm², we found that ˜35% of cells growing at the monomeric collagen either detached or displayed PI stain, showing still is a lower level of photodamage compared to ˜70% of detached and PI positive cells growing on fibrillar collagen substrates. Thus, we concluded that collagen fibrils generating second harmonic signal could contribute to the excitation of Chlorin e6 for photo-treatment.

Considering the significant SHG nonlinear-optical conversion demonstrated here, it is important to note that many reports on the conventional TPA-induced PDT utilizing pico- or femto-second Ti-Sapphire lasers at the excitation wavelength in the range of 750-850 nm, could also have contributions from the SHG signal at ˜400 nm generated by fibrillar collagen in malignant tissues.

Experimental demonstration of the application of a series of new nonlinear-optical conversion mechanisms—resonance CARS, FWM, SHG for the enhancement of phototherapy efficiency was demonstrated. These mechanisms can be used for advanced triggering of PDT when intra- or extra-cellular native biomolecules are used for efficient nonlinear-optical upconversion of incident IR beams for single photon excitation of PDT drug.

This technology can be used complementary to the conventional two-photon PDT, accumulating treatment benefits from all nonlinear-optical mechanisms—TPA, CARS/FWM, SHG. A combination of such newly proposed nonlinear-optical excitation techniques with already well developed two-photon PDT technology, enables for highly selective, high-resolution, deep penetrating, lower radiation threshold enhanced PDT treatment. It is considered that an implementation of the proposed concept will improve the most critical parameters—effective depth of tissue accessible and reduce concentration of photosynthesizers required for efficient PDT process.

For in vivo application, the device has to have an optical system with tight focusing of output waves and large working distance to allow deep penetration of light in tissue. The most close to ready-for-application device could be the one described in PNAS 102 (46) 16807-16812 (2005). Modification of objective lens will allow using it for PDT treatment of internal tumors in vivo.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

1) A method of generating singlet oxygen and/or reactive oxygen species in a localized volume of an individual comprising:
a) administering to the individual a photosensitizing agent having an absorption band maximum in the wavelength range of from 350 to 700 nm; and
b) exposing the individual to incident coherent pulsed electromagnetic energy having a wavelength between 700 nm and 1.4 microns and a repetition rate of from 100 MHz to 1 Hz, wherein a secondary electromagnetic energy having a wavelength of 350 to 700 nm, is produced in the localized volume of the individual, and the photosensitizing agent is excited by the secondary electromagnetic energy resulting in generation of singlet oxygen and/or reactive oxygen species in at least a portion of the localized volume of the individual.

2) The method of claim 1, wherein the incident coherent electromagnetic energy is provided by a single laser.

3) The method of claim 1, wherein the incident coherent electromagnetic energy is provided by a first laser providing a first incident coherent electromagnetic energy and a second laser providing a second incident coherent electromagnetic energy and the first coherent electromagnetic energy and the second coherent electromagnetic energy are synchronized in time and overlapped in space.

4) The method of claim 1, wherein the photodynamic therapy compound is selected from porphyrins, bacterioporphyrins, corrins, chlorins, bacteriochlorines, bacteriochlorophylls, corphins, phtalocyanins, azadipyrromethenes, and complexes thereof.

5) The method of claim 1, wherein the incident coherent light has an average power density of from 1×10⁶ W/cm² to 5×10⁷ W/cm².

6) The method of claim 1, wherein the localized volume of the individual at least 50 microns below a surface of the individual exposed to the ambient atmosphere, a mucosal surface of the individual, a surface of the respiratory tract of the individual, a surface of the gastrointestinal tract of the individual, or a surface of the reproductive tract of the individual.

7) The method of claim 2, wherein the photodynamic therapy compound is selected from porphyrins, bacterioporphyrins, corrins, chlorins, bacteriochlorines, bacteriochlorophylls, corphins, phtalocyanins, azadipyrromethenes, and complexes thereof.

8) The method of claim 2, wherein the incident coherent light has an average power density of from 1×10⁶ W/cm² to 5×10⁷ W/cm².

9) The method of claim 2, wherein the localized volume of the individual at least 50 microns below a surface of the individual exposed to the ambient atmosphere, a mucosal surface of the individual, a surface of the respiratory tract of the individual, a surface of the gastrointestinal tract of the individual, or a surface of the reproductive tract of the individual.

10) The method of claim 3, wherein the photodynamic therapy compound is selected from porphyrins, bacterioporphyrins, corrins, chlorins, bacteriochlorines, bacteriochlorophylls, corphins, phtalocyanins, azadipyrromethenes, and complexes thereof.

11) The method of claim 3, wherein the incident coherent light has an average power density of from 1×10⁶ W/cm² to 5×10⁷ W/cm².

12) The method of claim 3, wherein the localized volume of the individual at least 50 microns below a surface of the individual exposed to the ambient atmosphere, a mucosal surface of the individual, a surface of the respiratory tract of the individual, a surface of the gastrointestinal tract of the individual, or a surface of the reproductive tract of the individual.

13) The method of claim 4, wherein the incident coherent light has an average power density of from 1×10⁶ W/cm² to 5×10⁷ W/cm².

14) The method of claim 4, wherein the localized volume of the individual at least 50 microns below a surface of the individual exposed to the ambient atmosphere, a mucosal surface of the individual, a surface of the respiratory tract of the individual, a surface of the gastrointestinal tract of the individual, or a surface of the reproductive tract of the individual.

15) The method of claim 5, wherein the localized volume of the individual at least 50 microns below a surface of the individual exposed to the ambient atmosphere, a mucosal surface of the individual, a surface of the respiratory tract of the individual, a surface of the gastrointestinal tract of the individual, or a surface of the reproductive tract of the individual.
